(19) United States
(12) Patent Application Publication
(10) Pub. No.: US 2006/0210505 A1
(43) Pub. Date: Sep. 21, 2006

(54) Multi-phase personal care composition comprising visually distinct phases

(76) Inventors: Mannie Lee Clapp, Mason, OH (US); Cheyne Pohiman Thomas, Independence, KY (US); Christopher Dean Putman, West Chester, OH (US); Colin Michael Mchugh, Mason, OH (US); Laurence Robert Bramlage, Batavia, OH (US); John Christopher Wesner, Liberty Twp, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/378,686

(22) Filed: Mar. 17, 2006

Related U.S. Application Data
(60) Provisional application No. 60/663,778, filed on Mar. 21, 2005.

(51) Int. Cl. A61K 8/02

(52) U.S. Cl. 424/70.1; 424/401

(57) ABSTRACT

Multi-phase personal care compositions comprise a first phase and a second phase, wherein said first and second phases form a visually distinct pattern. The compositions are intended for moisturizing or conditioning skin or hair and comprise less than about 10%, by weight of the multi-phase personal care composition, of surfactant. Methods of moisturizing or conditioning skin or hair comprise the steps of wetting the skin or hair, applying the multi-phase personal care composition to the skin or hair, and rinsing the multi-phase personal care composition from the skin or hair. A process for making the multi-phase personal care composition comprises forming a first phase by premixing an oil/emollient benefit agent and oil-soluble colorant and then combining the premix with additional optional ingredients.

### CROSS-REFERENCE TO RELATED APPLICATIONS

This application claims the benefit of Provisional Application Ser. No. 60/663,778, filed Mar. 21, 2005.

### FIELD OF THE INVENTION

The present invention relates to the field of personal care compositions for improving appearance and feel of keratinous surfaces. More specifically, the present invention relates to rinsable personal care compositions that provide excellent appearance, moisturization, and conditioning of skin or hair.

### BACKGROUND OF THE INVENTION

Personal care compositions are well known and widely used. These compositions have long been employed to cleanse and/or moisturize/condition skin or hair, deliver actives, hide imperfections and to reduce the oiliness/shine associated with sebum.

One form of personal care composition gaining in popularity is a body lotion product that is applied to wet skin while showering or bathing and then rinsing the skin with water (i.e. an “in-shower body lotion” product). With this type of personal care composition, a consumer can more conveniently apply body lotion while in the shower, as opposed to the common practice of applying body lotion after getting out of the shower. Such compositions tend to have a milky white, opaque, homogeneous appearance and consist of a single visible phase. These compositions are typically packaged in an opaque container such that the composition is not visible through the walls of the container as it sits on a store shelf.

The aesthetic appearance of a product, especially of a personal care product, as it sits on a store shelf can be an important factor in a consumer's initial product purchase decision. Since the store shelves tend to be crowded with personal care products, it can be important for a personal care product to have an aesthetic appearance that is different from other products to help differentiate it on the store shelves. This can be especially important for a new type or category of personal care product, such as an in-shower body lotion product, to help consumer's recognize the new type or category of product on the store shelf. It has thus been desired to develop a personal care composition, especially an in-shower body lotion product, which exhibits a different aesthetic appearance to consumers as the product sits on a store shelf.

### SUMMARY OF THE INVENTION

The present invention relates to a multi-phase personal care composition comprising a first phase and a second phase, wherein the first and second phases form a visually distinct pattern, and wherein the composition comprises less than about 10%, by weight of the multi-phase personal care composition, of surfactant. The multi-phase personal care compositions of the present invention are generally intended to moisturize or condition skin or hair. The phases of the present compositions are combined to provide an aesthetic visually distinct pattern, which can be visible to a consumer when the composition is packaged in a transparent or translucent container as it sits on a store shelf.

In one embodiment, the present multi-phase personal care composition comprises an opaque first phase comprising an oil/emollient benefit agent and a colorant, the opaque first phase having a viscosity of at least about 1,000 Pa·s; and an opaque second phase having a viscosity of at least about 1,000 Pa·s; wherein the first phase and the second phase form a visually distinct pattern; and wherein the multi-phase personal care composition comprises less than about 10%, by weight of the multi-phase personal care composition, of surfactant.

The present invention further relates to a process of manufacture that comprises the steps of making a first phase comprising an oil/emollient benefit agent and an oil-soluble colorant, wherein the oil/emollient benefit agent and the oil-soluble colorant are mixed to form a premix, wherein the premix is combined with additional optional ingredients to form the first phase; making a second phase; and combining the first phase and the second phase to form the multi-phase personal care composition of the present invention; wherein the multi-phase personal care composition comprises less than about 10%, by weight of the multi-phase personal care composition, of surfactant. Forming a premix of the colorant and the oil/emollient benefit agent tends to inhibit migration of the colorant from one phase of the composition to another.

The present invention further relates to a method of moisturizing or conditioning skin or hair, comprising the steps of wetting skin or hair with water; applying to skin or hair a multi-phase personal care composition of comprising: a first phase and a second phase; wherein the phases form a visually distinct pattern and wherein the multi-phase personal care composition comprises less than about 10%, by weight of the multi-phase personal care composition, of surfactant; and then rinsing the multi-phase personal care composition from the skin or hair with water. The present invention thus provides a product that not only has an aesthetically different appearance from conventional personal care compositions, but also can be used in the shower to moisturize or condition skin or hair.

### DETAILED DESCRIPTION OF THE INVENTION

By the term “multi-phased” or “multi-phase” as used herein, is meant that the phases of the present multi-phase personal care composition occupy separate but distinct physical spaces inside the package in which they are stored, and are in direct physical contact with one another (i.e., they are not separated by a physical barrier and they are not emulsified or mixed together to any significant degree). The phases of the present “multi-phased” personal care composition are combined to form a visually distinct pattern. The pattern results from the combination or homogenization of phases of the “multi-phase” composition into a single compartment container. The visually distinct patterns include, but are not limited to, the following examples: striped, marbled, rectilinear, interrupted striped, check, mottled, veined, clustered, speckled, geometric, spotted, ribbons, helical, swirl, arrayed, variegated, textured, grooved, ridged, waved, sinusoidal, spiral, twisted, curved, cycle, streaks, striated, contoured, anisotropic, laced, weave or woven, basket weave, spotted, and tessellated. Preferably the pattern is selected from the group consisting of striped, geometric, marbled, and combinations thereof.

In one embodiment, the visually distinct pattern may be relatively uniform and even across the dimension of the package. Alternatively, the visually distinct pattern may be uneven, i.e. wavy, or may be non-uniform in dimension. The visually distinct pattern does not need to necessarily extend across the entire dimension of the package. The visually distinct pattern can comprise stripes, wherein the size of the stripes is at least about 0.1 mm in width and 10 mm in length, preferably at least about 1 mm in width and at least 20 mm in length. The phases may be various different colors.

The term “personal care composition” as used herein, refers to compositions intended for topical application to the skin or hair.

The term “phases” as used herein, refers to a domain or region of a composition having one average composition, as distinct from another region or domain having similar or different average composition, wherein the distinct domains are visible to the naked eye. This would not preclude the distinct regions or domains from comprising two similar phases where one phase could comprise colorants, and various optional ingredients, hence a region or domain of a similar or different average composition.

The term “stable” as used herein, unless otherwise specified, refers to compositions that maintain at least two “separate” phases when sitting in physical contact at ambient conditions for a period of at least about 180 days. By “separate” is meant that there is substantially no mixing of the phases, observable to the naked eye, prior to dispensing of the composition.

Multi-Phase Personal Care Composition

The multi-phase personal care compositions of the present invention comprise at least two visually distinct phases. By incorporating a different colorant in each phase, or leaving one phase without a colorant, a visually distinct pattern can be formed by the phases in the composition.

First Phase

A first phase of the present multi-phase personal care compositions will generally comprise an oil/emollient benefit agent and other optional ingredients.

Oil/Emollient Benfefit Agents

Oil/emollient benefit agents are incorporated in a first phase of the present multi-phase personal care compositions to provide a moisturization/conditioning benefit to skin or hair. Oil/emollient benefit agents are typically incorporated in a phase of the present multi-phase personal care compositions at a level of at least about 0.1%, preferably at least about 1%, more preferably at least about 5%, more preferably at least about 7%, more preferably at least about 10%, and even more preferably at least about 18%, by weight of the phase of the multi-phase personal care composition. Oil/emollient benefit agents are typically incorporated in a phase of the present multi-phase personal care compositions at a level of less than about 99%, preferably less than about 70%, more preferably less than about 60%, and even more preferably less than about 50%, by weight of the phase of the multi-phase personal care composition.

The oil/emollient benefit agents incorporated in the present compositions encompass a variety of suitable materials. Examples of suitable oil/emollient benefit agents include triglycerides, hydrocarbon oils, polyesters, silicone oils, and mixtures thereof.

One class of useful oil/emollient benefit agent is the triglycerides and modified triglycerides. These include vegetable oils such as jojoba, soybean, canola, sunflower, safflower, rice bran, avocado, almond, olive, sesame, persic, castor, coconut, and mink oils. Synthetic triglycerides can also be employed. Modified triglycerides include materials such as ethoxylated and maleated triglyceride derivatives provided they are liquids. Proprietary ester blends such as those sold by Finetex as Finsolv are also suitable, as is ethylhexanoic acid glyceride.

Another type of oil/emollient benefit agent suitable herein is liquid polyester formed from the reaction of a dicarboxylic acid and a diol. Examples of polyesters suitable for the present invention are the polyesters marketed by ExxonMobil under the trade name PURESYN ESTER.

Another class of oil/emollient benefit agents suitable for the present invention is liquid and semi-solid hydrocarbons. These include linear and branched oils such as liquid paraffin, squalene, squalane, mineral oil, low viscosity synthetic hydrocarbons such as polyalphaolefin sold by ExxonMobil under the trade name of PURESYN PAO and polybutene under the trade name PANALANE or INDOPOL. Light (low viscosity) highly branched hydrocarbon oils are also suitable. Petrolatum is a suitable oil/emollient benefit agent and is a unique hydrocarbon material. The semi-solid nature of petrolatum can be controlled both in production and by the formulator through blending with other oils.

Another class of useful oil/emollient benefit agents is silicone based. They include linear and cyclic polydimethyl siloxane, organo functional silicones (alkyl and alkyl aryl), and amino silicones.

Other suitable oil/emollient benefit agents include benefit agents (e.g. skin compatible oils) as disclosed in US 2003/0054019 A1; US 2003/0180243 A1; US 2003/0190296 A1; US 2004/0234558 A1; US 2004/0235691 A1; US 2004/0234469 A1; US 2004/0234467 A1; US 2004/0234470 A1; US 2004/0234468 A1; U.S. Pat. No. 6,534,456; U.S. Pat. No. 6,534,457; U.S. Pat. No. 6,645,511; and U.S. Pat. No. 6,716,440.

Preferred oil/emollient benefit agents include high modulus lipids as disclosed in U.S. Provisional Application Ser. No. 60/658,687, filed Mar. 4, 2005 (Case 9933P).

Auxiliary Benefit Agents

A first phase of the present multi-phase personal care compositions can optionally further comprise auxiliary benefit agents. Suitable auxiliary benefits agents include a variety of materials, such as those described in U.S. Provisional Application Ser. No. 60/658,687, filed Mar. 4, 2005 (Case 9933P), including: desquamation actives (e.g. a combination of sulfhydryl compounds or salicylic acid and zwitterionic surfactants); anti-acne actives (e.g. resorcinol, salicylic acid, benzoyl peroxide, erthyromycin, zinc); anti-wrinkle actives (e.g. alpha or beta hydroxy acids, niacinamide, retinol, retinol esters); anti-oxidants (e.g. ascorbic acid and its salts, gallic acid and its alkyl esters); chelators (e.g. furildioxime, furilmonoxime); flavanoids (e.g. flavanones, chalcones, flavones, coumarins, chromones); anti-inflammatory agents (e.g. corticosteroids such as hydrocortisone); anti-cellulite agents (e.g. xanthine compounds such as caffeine, theophylline, theobromine, aminophylline); topical anesthetics (e.g. benzocaine, lidocaine); tanning actives (e.g. dihydroxyacetone); skin lightening agents (e.g. kojic acid, arbutin, titanium dioxide, zinc oxidekojic acid, arbutin, ascorbic acid and derivatives thereof (e.g., magnesium ascorbyl phosphate or sodium ascorbyl phosphate), and extracts (e.g., mulberry extract, placental extract) as well as titanium dioxide and zinc oxide); skin soothing and healing actives (e.g. panthenoic acid derivatives, aloe vera, allantoin); sunscreen actives (e.g. p-aminobenzoic acid and its salts and derivatives, cinnamic acid derivatives); and visual skin enhancers. These auxiliary benefit agents can be used in a wide variety of combinations in the phases of the present compositions.

Other examples of suitable auxiliary benefit agents include: (a) humectants that can be used to retain water in the skin or hair such as glycerin, sorbitol, glycols, polyols, urea, water-soluble polymers; (b) lipid barrier repair agents that can be useful for strengthening and replenishing the stratum corneum's barrier lipids, such as cholesterol, cholesterol esters (e.g. cholesterol isostearate), ceramides, and pseudoceramides; and (c) vitamins that can be used to strengthen the skin such as vitamin A, vitamin B, vitamin E, vitamin alkyl esters such as vitamin C alkyl esters.

Auxiliary benefit agents are optionally incorporated in a first phase of the present compositions at a typical level of from about 0.01% to about 50%, by weight of the phase of the multi-phase personal care composition. The level will typically depend on the material and are described more fully in U.S. Provisional Application Ser. No. 60/658,687, filed Mar. 4, 2005 (Case 9933P).

Structurants

A first phase of the present multi-phase personal care compositions can optionally further comprise a structurant. A structurant can help provide the oil/emollient benefit agent (and the phase itself) with desirable rheological properties. The amount of structurant will vary depending on the oil/emollient benefit agent and the structurant, but in general, the structurant will be incorporated in a phase of the present compositions at a level of less than about 75%, more preferably less than about 60%, and still more preferably less than about 50%, by weight of the phase of the present composition.

Structurants can tend to form 3-dimensional networks to build up the viscosity of the oil/emollient benefit agents. Such structured oil phases, i.e., built with the 3-dimensional network, can be desirable for use as in-shower body lotion compositions used in bathing. These structured oils can tend to deposit and be retained effectively on wet skin and retained after rinsing and drying to provide long-lasting after wash skin benefit without causing a too oily/greasy wet and dry feel. It is believed that the highly desirable in-use and after-use properties of such structured oils are due to their shear thinning rheological properties and the weak structure of the network. Due to its high low-shear viscosity, the 3-dimensional network structured oil can stick and retain well on the skin during application of the in-shower body lotion. After being deposited on the skin, the network yields easily during rubbing due to the weak structuring of the crystal network and its lower high-shear viscosity.

The structurant can be either an organic or inorganic structurant. Examples of organic structurants suitable for the invention can be selected from the group consisting of natural or modified fats, fatty acid, fatty amine, fatty alcohol, natural and synthetic waxes, block copolymers, and mixtures thereof. Suitable fatty acids include C₁₀-C₂₂ fatty acids such as lauric acid, myristic acid, oleic acid, isostearic acid, linoleic acid, linolenic acid, ricinoleic acid, elaidic acid, arichidonic acid, myristoleic acid, palmitoleic acid, and the like. Suitable block polymers for this application can be those sold under the name KRATON by Shell. Inorganic structuring agents can be selected from the group consisting of hydrophobically modified silica, hydrophobically modified clay, and mixtures thereof. Non-limiting examples of inorganic structurants are BENTONE 27V, BENTONE 38V or BENTONE GEL MIO V from Rheox; and CAB-O-SIL TS720 or CAB-O-SIL M5 from Cabot Corporation.

The structurant can be a natural or synthetic crystalline wax. Mineral, animal or plant (vegetable) waxes are all described as natural waxes. Synthetic waxes are described as those waxes that have been synthetically polymerized from raw materials or chemically modified natural waxes.

Among the natural crystalline waxes which may be used are petroleum based waxes such as paraffins and microcrystalline wax. Molecular weights of paraffin waxes generally range from 360 to 420 (26 to 30 carbon atoms), although versions with longer chains (molecular weights up to 600) are available. Typical melting points are 126-134° F. (52-57° C.), the high molecular weight versions have melting points near 170° F. (77° C.). Paraffin waxes are brittle and the addition of oil weakens the structure (lowers the tensile strength).

Microcrystalline waxes (MC) melting points are 145 to 195° F. (63-91° C.). The crystals of MC wax are small and irregular and consist of several types: plates, malcrystalline and needle. Animal waxes can be obtained from such things as bees, insects or whales. These waxes include but are not limited to beeswax, Chinese wax, shellac wax, spermaceti and wool wax. Plant waxes can be derived from beans, leaves and berries. Plant or vegetable waxes can include bayberry, candelilla, carnauba, cotton, esparto, fir, Japan, ouricury, palm, rice-oil, sugar cane, ucuhuba and cocoa butter.

Among synthetic crystalline waxes which may be used are crystalline polymers such as polyethylene, Fischer-Tropsch waxes such as polymethylene, chemically modified waxes, polymerized alpha olefins and synthetic animal waxes. For example, siliconyl beeswax may be used which is beeswax that has been chemically modified.

In addition, structurant may be a natural or synthetic hydrogenated oils or fats. In addition some fatty acids and fatty alcohols can be used as structurant as well as salts of fatty acids and hydroxy fatty acids.

Hydrogenated oils can be hydrogenated vegetable oils, hydrogenated coconut oil, hydrogenated palm kernel oil, hydrogenated rapeseed oil, castorwax and many others.

Crystalline long chain fatty acids and long chain fatty alcohols can also be used to structure benefit agents. Examples of fatty acids are myristic acid, palmitic acid, stearic acid, arachidic acid and behenic acid. Examples of fatty alcohols are palmityl alcohol, stearyl alcohol, arachyl alcohol and behenyl alcohol.

Another suitable structurant is trihydroxystearin (available under the tradename THIXCIN from Rheox).

Other suitable structurants are described in US 2003/0054019 A1; US 2003/0180243 A1; US 2003/0190296 A1; US 2004/0234558 A1; US 2004/0235691 A1; US 2004/0234469 A1; US 2004/0234467 A1; US 2004/0234470 A1; US 2004/0234468 A1; U.S. Pat. No. 6,534,456; U.S. Pat. No. 6,534,457; U.S. Pat. No. 6,645,511; and U.S. Pat. No. 6,716,440.

Esters

A first phase of the compositions of the present invention can optionally further comprise esters. Esters can serve to enhance the spreadability of the oil/emollient benefit agents and to reduce the tack typically associated with such materials. The esters can be premixed with the structured oil phase, or they can be added separately while the product is warm, or when the product is cooling.

The esters of the present invention may include ester oils and as the name implies, ester oils comprise at least one ester group in the molecule. One type of common ester oil useful in the present invention are the fatty acid mono and polyesters such as cetyl octanoate, octyl isonanoanate, myristyl lactate, cetyl lactate, isopropyl myristate, myristyl myristate, diisopropyl sebacate, diisotearyl malate, isostearyl neopentanoate, isopropyl palmitate, isopropyl adipate, butyl stearate, decyl oleate, glycerol monostearate, glycerol distearate, glycerol tristearate, alkyl lactate, alkyl citrate and alkyl tartrate; sucrose ester and polyesters, sorbitol ester, and the like.

The ester may be selected from the group consisting of a di-ester, tri-ester, tetra-ester, a branched ester, a dimer and mixtures thereof. Non-limiting examples of diesters may include diisopropyl adipate or diisopropyl sebacate. Non-limiting examples of branched ester may include ethylhexyl isononanoate. Non-limiting examples of dimers may include diisopropyl dimer dilinoleate. Non-limiting examples of tetraesters include pentaerythritol esters.

The ester's individual fatty groups will preferably comprise no more than 20 carbons, even more preferably no more than 17 carbons, even more preferably, no more than 14, and most preferably no more than 12. Preferably, the total number of carbon atoms in the ester will be less than 30, even more preferably less than 25, even more preferably less than 23.

Preferably, the ratio of oil/emollient benefit agent to ester is no more than about 50:1, more preferably no more than about 30:1, even more preferably no more than about 20:1, even more preferably no more than about 10:1, and most preferably no more than about 2:1. Preferably, the ratio of oil/emollient benefit agent to ester can comprise at least about 1:15, even more preferably at least about 1:10, even more preferably no more than about 1:8, even more preferably no more than about 1:4, even more preferably no more than about 1:2, and most preferably no more than about 1:1.

Thickening Agents

A first phase of the compositions of the present invention, in some embodiments, can optionally further comprise one or more thickening agents. Because different thickening agents thicken with different efficiencies, it is difficult to provide an accurate compositional range, however, when present, the present compositions preferably comprise no more than about 10%, more preferably no more than about 8%, and still more preferably no more than about 7%, by weight of the phase of the personal care composition. When present, the thickening agent preferably comprises at least about 0.01%, more preferably at least about 0.05%, and still more preferably at least about 0.1%, by weight of the phase of the personal care composition. It can often be useful to blend different thickening agents together to generate an optimal stability and rheology profile.

Non-limiting examples of thickening agents useful herein include carboxylic acid polymers such as carbomers (including those commercially available under the tradename CARBOPOL® 900 series from B.F. Goodrich; e.g., CARBOPOL® 954) and the Luvigel series from BASF. Other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as CARBOPOL® 1342, CARBOPOL® 1382, CARBOPOL Ultrez 21, PEMULEN TR-1, and PEMULEN TR-2, from B.F. Goodrich.

Other non-limiting examples of thickening agents include crosslinked polyacrylate polymers including both cationic and nonionic polymers.

Still other non-limiting examples of thickening agents include the polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the tradename SEPIGEL 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

Another non-limiting class of thickening agents useful herein are polysaccharides. Non-limiting examples of polysaccharide gelling agents include those selected from cellulose, and cellulose derivatives. Preferred among the alkyl hydroxyalkyl cellulose ethers is the material given the CTFA designation cetyl hydroxyethylcellulose, which is the ether of cetyl alcohol and hydroxyethylcellulose, sold under the tradename NATROSEL® CS PLUS from Aqualon Corporation (Wilmington, Del.). Other useful polysaccharides include scleroglucans which are a linear chain of (1-3) linked glucose units with a (1-6) linked glucose every three units, a commercially available example of which is CLEAROGEL™ CS 11 from Michel Mercier Products Inc. (Mountainside, N.J.).

Another non-limiting class of thickening agents useful herein are gums. Non-limiting examples of gums useful herein include hectorite, hydrated silica, xantham gum, and mixtures thereof.

Another non-limiting class of thickening agents useful herein are the modified starches. Acrylate modified starches such as WATERLOCK® from Grain Processing Corporation may be used. Hydroxypropyl starch phosphate, tradename STRUCTURE XL from National Starch, is another example of a useful modified starch, and other useful examples include ARISTOFLEX HMB (Ammonium Acrylodimethyltaruate/Beheneth-25 Methacrylate Crosspolymer) from Clariant and cationic stabylens.

Surfactant

The multi-phase personal care compositions of the present invention can optionally comprise surfactants. The surfactant, if present, can be included in one or more phases of the composition. Non-limiting examples of suitable surfactants include anionic surfactants, nonionic surfactants, amphoteric surfactants, cationic surfactants, and mixtures thereof. Examples of suitable surfactants are described in detail in U.S. Provisional Application Ser. No. 60/658,687, filed Mar. 4, 2005 (Case 9933P).

The present compositions will preferably comprise only relatively low levels of surfactants. As such, the present compositions will typically comprise less than about 10%, more preferably less than about 5%, more preferably less than about 1%, and even more preferably less than about 0.5%, by weight of the multi-phase personal care composition, of surfactant.

Emulsifiers

A first phase of the multi-phase personal care compositions of the present invention can optionally further comprise an emulsifier. A wide variety of emulsifiers are useful herein. The composition of the present invention can comprise a sufficient amount of one or more emulsifiers to emulsify the oil/emollient benefit agent to yield an appropriate particle size and good application properties on wet skin.

Non-limiting examples of emulsifiers suitable for the present compositions inlcude: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

In addition, there are several commercial emulsifier mixtures that are useful in some embodiments. Examples include but are not limited to PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N—N-Dimethyl,N—C(16-18) Acyl Derivatives, Chlorides) from ISP; POLAWAX NF (Emulsifying wax NF), INCROQUAT BEHENYL TMS (behentrimonium sulfate and cetearyl alcohol) from Croda; POLAWAX Pastilles (blend of stearyl alcohol, cetyl alcohol, and Polysorbate 60) from Croda; and EMULLIUM DELTA (cetyl alcohol, glyceryl stearate, peg-75 stearate, ceteth-20 and steareth-20) from Gattefosse. The emulsifier systems used in conjunction with the present invention should not be limited to those disclosed in the specification, one skilled could use any emulsifier system know in the art keeping in mind the personal care composition of the present invention. These systems can be used as provided or the individual components can be used in any combination to produce the desired effect.

Emulsifiers are typically included in the present compositions at a level of at least about 0.01%, and less than about 10%, preferably less than about 5%, and more preferably at least about 1%, and even more preferably less than about 0.5%, by weight of the phase of the multi-phase personal care composition.

Antimicrobial Agents

A first phase of the multi-phase personal care compositions of the present invention can optionally further comprise an antimicrobial agent, preferred concentrations of which range from about 0.001% to about 10%, more preferably from about 0.01% to about 5%, and still more preferably from about 0.05% to about 2%, by weight of the phase of the present composition.

Non-limiting examples of antimicrobial actives for use herein includes β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione, clotrimazole, and combinations thereof.

Colorants

A first phase of the present multi-phase personal care composition can optionally further comprise a colorant. A wide variety of colorants can be used, such as water-soluble colorants, oil-soluble colorants, or particle pigments. In one embodiment of the present invention, the first phase comprises an oil-soluble colorant. An example of an oil-soluble colorant is D&C Violet 2. Examples of particle pigments include interference pigments, especially hydrophobically modified interference pigments, which are described in U.S. Provisional Application Ser. No. 60/658,687, filed Mar. 4, 2005 (Case 9933P).

Colorants are typically incorporated in the present compositions at a level of from about 0.001% to about 10%, preferably less than about 5%, more preferably less than about 1%, by weight of the phase of the multi-phase personal care composition.

Water

A first phase of the present multi-phase personal care compositions will typically comprise water. Water is typically incorporated in a first phase of the present compositions at a level of at least about 10%, preferably at least about 20%, more preferably at least about 30%, even more preferably at least about 40%, and even more preferably at least about 50%, by weight of the phase of the multi-phase personal care composition. Water is typically incorporated in the present compositions at a level of less than about 90%, preferably less than about 80%, more preferably less than about 75%, and even more preferably less than about 70%, by weight of the phase of the multi-phase personal care composition. In one embodiment, the present multi-phase personal care composition comprises at least about 40%, preferably at least about 50%, and more preferably at least about 60%, by weight of the multi-phase personal care composition, of water.

Other Optional Ingredients

The CTFA Cosmetic Ingredient Handbook, Second Edition (1992) describes a wide variety of non-limiting cosmetic and pharmaceutical ingredients commonly used in the personal care industry, which are suitable for use in a first phase of the compositions of the present invention as other optional ingredients. A preferred optional ingredient is fragrance.

Second Phase

The multi-phase personal care compositions of the present invention further comprise a second phase that is visually distinct from the first phase. When the first and second phases are combined to form the present composition, a visually distinct pattern is created by the phases. The second phase of the present compositions can be comprised of similar, or the same, ingredients as the first phase. If the first phase comprises a colorant, the second phase will comprise a different colorant or no colorant, such that the second phase is visually distinct from the first phase. If the first phase does not comprise a colorant, the second phase will comprise a colorant such that the second phase is visually distinct from the first phase. In one embodiment, the second phase comprises substantially the same components as the first phase, except for utilizing a colorant (if the first phase does not comprise a colorant) or utilizing a different colorant (if the first phase does comprise a colorant).

Third, Fourth, et al. Phases

The present compositions can optionally further comprise a number of additional phases, e.g., a third phase, fourth phase, et al. These additional phases can be comprised of similar, or the same, ingredients as the first and/or second phases. These additional phases preferably comprise a different colorant such that the additional phases are visually distinct from the first and/or second phases.

Each of the phases of the present compositions can be opaque, transparent, or translucent. Aqueous gel phases are typically transparent or translucent. In one embodiment, the first phase is opaque and the second phase is opaque. In one embodiment, the present composition is free of an aqueous gel phase.

One or more phases of the present compositions are preferably dispersions, more preferably emulsions, such as an oil-in-water emulsion, a water-in-oil emulsion, or combinations thereof. In one embodiment, the present composition comprises a first phase that is an oil-in-water emulsion and a second phase that is an oil-in-water emulsion.

The phases of the present compositions preferably do not form “temporary emulsions” upon shaking. Temporary emulsions are formed when a multi-phase product is shaken and then the phases separate back out upon leaving the composition to sit without agitation.

The compositions of the present invention are preferably stable as defined herein, such that the visually distinct pattern formed by the phases is maintained as the product sits on a store shelf over a period of time.

In preferred embodiments, one or more phases of the present compositions have non-Newtonian or shear-thinning properties. It can be important for the phases of the present compositions to have non-Newtonian or shear-thinning properties such that the appropriate viscosity of each phase can be maintained while the composition is stored in a container and then upon dispensing from the container, the viscosity becomes lower to facilitate dispensing and spreading of the composition on skin or hair of the consumer.

Viscosity

Each of the phases of the present multi-phase compositions will generally have a viscosity of at least about 1,000 Pascal·seconds (“Pa·s”), preferably at least about 4,000 Pa·s, and more preferably at least about 8,000 Pa·s. The viscosity of each phase, or of the present multi-phase composition itself, is measured according to the Viscosity Measurement Method described below. The viscosities of the first and second phases of the present compositions preferably differ by no more than about 20%, preferably no more than about 15%, and more preferably no more than about 10%.

Package

The multi-phase personal care compositions of the present invention can be packaged in a variety of different containers. Since the phases of the present compositions form visually distinct patterns, it is preferred to package the compositions is a transparent or translucent container, as opposed to an opaque container, so that a consumer can see the visually distinct pattern through the container as it sits on a store shelf.

Preferred containers for the present multi-phase personal care compositions, such as “tottles” (which is a container comprising a bottle and a cap attached to the bottle, wherein the container is designed to rest or stand on its cap), are described in detail in U.S. application Ser. No. 11/067,443, filed Feb. 25, 2005 (Case 9926). For multi-phase personal care compositions of the present invention that are used in the shower, such as an in-shower body lotion product, a preferred container comprises a slit valve, as described in U.S. Provisional Application Ser. No. 60/626,133, filed Nov. 8, 2004 (Case 9633P2).

Process of Manufacture

A number of different processes for combining the phases described herein to form multi-phase compositions having visually distinct patterns are disclosed in the art. For example, a suitable process is described in US 2004/0219119 A1 (Case 9218). In this process, a first phase is stored in a first storage tank and a second phase is stored in a second storage tank. The first phase is pumped from the first storage tank into a static mixer. The second phase is pumped from the second storage tank into the same static mixer. The first and second phases are intermingled in the static mixer and pumped out of the static mixer and through a single nozzle for dispensing the phases into a container. The container is positioned below the nozzle and receives the phases while rotating on a spinning disc. The container is moved vertically with respect to the nozzle as the container is being filled with the phases. This process can result in a multi-phase personal care composition that has a marbled visual pattern formed by the phases of the composition.

Other suitable processes are described in U.S. Pat. No. 6,516,838, U.S. Pat. No. 6,367,519, and U.S. Pat. No. 6,213,166. In this process, a first phase is stored in a first storage tank and a second phase is stored in a second storage tank. The first phase is pumped from the first storage tank into a nozzle assembly having a plurality of nozzles. The second phase is pumped from the second storage tank into the nozzle assembly having a plurality of nozzles. The first and second phases are pumped through separate nozzles in the nozzle assembly for dispensing the phases into a container. Either the nozzle assembly is rotated with respect to the container, or the container is rotated with respect to the nozzle assembly, as the phases are separately dispensed into the container. Either the nozzle assembly is moved vertically with respect to the container, or the container is moved vertically with respect to the nozzle assembly as the container is being filled with the phases. This process can result in a multi-phase personal care composition that has a spiral or helical visual pattern formed by the phases of the composition.

Another suitable process is described in U.S. Pat. No. 4,159,028. In this process, a first phase is stored in a first storage tank and a second phase is stored in a second storage tank. The first phase is pumped from the first storage tank into a receiving cavity. The second phase is pumped from the second storage tank into the same receiving cavity. The first and second phases are pumped out of the receiving cavity and through a single nozzle for dispensing the phases into a container. The container is positioned below the nozzle at an angle and receives the phases while rotating on a spinning disc. The container is moved vertically with respect to the nozzle as the container is being filled with the phases. This process can result in a multi-phase personal care composition that has a generally swirled or marbled visual pattern formed by the phases of the composition.

In a preferred process of manufacture, the colorant (preferably an oil-soluble colorant) is premixed with the oil/emollient benefit agent before adding the premix to the rest of the ingredients of a particular phase. Premixing the colorant with the oil/emollient benefit agent, tends to prevent the colorant from migrating from one phase to another. As such, it is preferred to utilize an oil-soluble colorant in at least one of the phases.

Method of Use

The present invention further relates to a method of using the multi-phase personal care compositions described herein. The present compositions are preferably used to moisturize or condition skin or hair by applying the composition to wet skin or wet hair while in the shower or bath and then rinsing the composition from the skin or hair with water. The oil/emollient benefit agents are preferably deposited on the skin or hair as a result of this method, thereby moisturizing or conditioning the skin or hair.

In one embodiment, the present invention relates to a method of moisturizing or conditioning skin or hair, said method comprising the steps of: (a) wetting said skin or hair with water; (b) applying to said skin or hair a multi-phase personal care composition of comprising: (i) a first phase and a second phase, wherein said phases form a visually distinct pattern and wherein said multi-phase personal care composition comprises less than about 10%, by weight of said multi-phase personal care composition, of surfactant; and (c) rinsing said multi-phase personal care composition from said skin or hair with water.

Viscosity Measurement Method

The viscosity of a particular phase of the present multi-phase personal care composition, or of the entire composition itself, can be measured by applying a shear stress and measuring the shear rate using a rheometer, such as a TA Instruments AR2000 (TA Instruments, New Castle, Del., USA 19720). Viscosity is determined at different shear rates in the following manner.

For measurement, a 40 mm diameter parallel plate geometry with a gap of 1 mm is used unless there are particles greater than 0.25 mm, in which case a gap of 2 mm is used. The rheometer uses standard parallel plate conventions to report shear rate at the edge as shear rate of the test; and converts torque to stress using the factor 2/(πR³). Using a spatula, a sample comprising a small excess of the phase, or composition, to be measured is loaded onto the rheometer base plate which is at 25° C., the gap is obtained, and excess composition outside the top measurement geometry is removed, locking the top plate in position during the removal of excess sample. The sample is equilibrated to the base plate temperature for 2 minutes. As is known to one skilled in the art, the shear rate with a parallel plate geometry is expressed as the shear rate at the edge, which is also the maximum shear rate. The measurement is performed, which comprises ramping the stress from 0.1 Pa to 5,000 Pa over a 5.0 minute interval at 25° C., while collecting 100 viscosity data points, in a log progression. A shear rate of at least 500 1/seconds is obtained in the test, or the test is repeated with a fresh sample of the same component with a higher final stress value, maintaining the same rate of stress increase per time, until a shear rate of at least 500 1/sec is obtained during the measurement period. During the measurement, observe the sample to make certain the area under the top parallel plate is not evacuated of sample at any edge location during the measurement, or the measurement is repeated until a sample remains for the duration of the test. If after several trials a result cannot be obtained due to sample evacuation at the edge, the measurement is repeated leaving an excess reservoir of material at the edge (not scraping). If evacuation still cannot be avoided, a concentric cylinder geometry is used with a large excess of sample to avoid air pockets during loading. Selecting only viscosity data points between 0.2 and 2 Pa shear rates, a median viscosity is calculated. This initial median viscosity is separately multiplied by 5 and divided by 5 to define a range. Any viscosity points lying outside of this range (initial median/5 to initial median*5) are removed to define a smaller data set, and the median viscosity is recalculated from the smaller data set to report a viscosity value for the sample, expressed in units of Pa·s.

### EXAMPLE

The following is a non-limiting example of the multi-phase personal care composition of the present invention, which is an in-shower body lotion product.

FIRST PHASE

| | Amount |
|---|---|
| Ingredients | (By Weight of First Phase) |
| Petrolatum ^{a} | 22.0% |
| Diisopropyl Sebacate ^{b} | 3.5% |
| Hydroxypropyl Starch Phosphate ^{c} | 3.5% |
| Stearyl Alcohol, Cetyl Alcohol, | 2.4% |
| and Polysorbate 60 blend ^{d} | |
| Fragrance | 1.2% |
| Preservative ^{e} | 0.293% |
| Phenoxyethanol | 0.25% |
| Disodium EDTA ^{f} | 0.12% |
| Water | Balance to 100% |

SECOND PHASE

| | Amount |
|---|---|
| Ingredients | (By Weight of Second Phase) |
| Petrolatum ^{a} | 22.0% |
| Colorant ^{g} | 0.003% |
| Diisopropyl Sebacate ^{b} | 3.5% |
| Hydroxypropyl Starch Phosphate ^{c} | 3.5% |
| Stearyl Alcohol, Cetyl Alcohol, | 2.4% |
| and Polysorbate 60 blend ^{d} | |
| Fragrance | 1.2% |
| Preservative ^{e} | 0.293% |
| Phenoxyethanol | 0.25% |
| Disodium EDTA ^{f} | 0.12% |
| Water | Balance to 100% |

^{a} Commercially available from Crompton Witco under the tradename G-2180 Petrolatum.
^{b} Commercially available from Noveon under the tradename SCHERCEMOL DIS.
^{c} Commercially available from National Starch under the tradename STRUCTURE XL.
^{d} Commercially available from Croda under the tradename POLAWAX Pastilles.
^{e} Commercially available from Lonza under the tradename GLYDANT PLUS Liquid.
^{f} Commercially available from Akzo Nobel under the tradename DISSOLVINE NA2-S.
^{g} Commercially available under the tradename D&C Violet 2.

The first and second phases of the multi-phase personal care composition exemplified above are both opaque. The viscosity of the first phase of the in-shower body lotion is about 8,500 Pa·s. The viscosity of the second phase of the in-shower body lotion is about 8,000 Pa·s. The first and second phases are both oil-in-water emulsions and are both non-Newtonian. The first and second phases are combined as described below and form a visually distinct striped pattern.

The multi-phase personal care composition exemplified above, which is an in-shower body lotion, is made by separately making the first phase and the second phase, and then combining them according to the process described in US 2004/0219119 A1 (Case 9218) to form the finished multi-phase personal care composition.

First Phase

The first phase is made according to the following procedure. Add about 300 grams of water to a first beaker and heat the water to about 85-90° C. In a second beaker, add about 66 grams of melted petrolatum and heat to about 85-90° C. Add about 7.2 grams of POLAWAX to the second beaker and mix. Add about 10.5 grams of STRUCTURE XL to the second beaker and mix. Take about 199.892 grams of heated water from the first beaker, add it to the second beaker, and mix. Add about 0.36 grams of Disodium EDTA to the second beaker. Add about 0.75 grams of Phenoxyethanol to the second beaker and mix. Move the second beaker to a water bath, continue mixing, and adjust the temperature of the contents of the second beaker to about 47° C. Add about 10.5 grams of SCHERCEMOL DIS to the second beaker at about 47° C. Add about 1.198 grams of GLYDANT PLUS liquid to the second beaker at about 46° C. and mix. Add about 3.6 grams of perfume to the second beaker at about 45° C. and mix. Cool the contents of the second beaker while mixing and then empty the contents into a first storage tank.

Second Phase

The second phase is made according to the following procedure. Add about 300 grams of water to a third beaker and heat the water to about 85-90° C. In a fourth beaker, add about 66 grams of melted petrolatum and heat to about 85-90° C. Add about 0.008 grams of colorant to the fourth beaker and mix until the colorant is dissolved in the petrolatum. Add about 7.2 grams of POLAWAX to the fourth beaker and mix. Add about 10.5 grams of STRUCTURE XL to the fourth beaker and mix. Take about 199.884 grams of heated water from the third beaker, add it to the fourth beaker, and mix. Add about 0.36 grams of disodium EDTA to the fourth beaker. Add about 0.75 grams of phenoxyethanol to the fourth beaker and mix. Move the fourth beaker to a water bath, continue mixing, and adjust the temperature of the contents of the fourth beaker to about 47° C. Add about 10.5 grams of SCHERCEMOL DIS to the fourth beaker at about 47° C. Add about 1.198 grams of GLYDANT PLUS liquid to the fourth beaker at about 46° C. and mix. Add about 3.6 grams of perfume to the fourth beaker at about 45° C. and mix. Cool the contents of the fourth beaker while mixing and then empty the contents into a second storage tank.

Combining the First and Second Phases

The first and second phases combined to form a multi-phase personal care composition according to a process similar to that described in US 2004/0219119 A1 (Case 9218), except that a static mixer is not utilized. The process is also similar to that described in U.S. Pat. No. 4,159,028. The first phase is pumped from the first storage tank into a receiving cavity. The second phase is pumped from the second storage tank into the same receiving cavity. The first and second phases are then pumped out of the receiving cavity and through a filling nozzle to form the multi-phase personal care composition. A plastic bottle, or other package, is placed directly underneath the filling nozzle to receive the multi-phase personal care composition from the filling nozzle. The plastic bottle is positioned on a bottle holding stand that lowers and rotates the bottle during filling. As the multi-phase personal care composition flows from the filling nozzle, the bottle holding stand lowers and rotates the bottle during filling at about 250 rpm. When the bottle is filled with the multi-phase personal care composition, the process is complete. The phases in the multi-phase personal care composition form a visually distinct striped pattern.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-phase personal care composition comprising:
an opaque first phase comprising an oil/emollient benefit agent and a colorant,
said opaque first phase having a viscosity of at least about 1,000 Pa·s; and
an opaque second phase having a viscosity of at least about 1,000 Pa·s;
wherein said first phase and said second phase form a visually distinct pattern; and
wherein said multi-phase personal care composition comprises less than about 10%, by weight of said multi-phase personal care composition, of surfactant.

2. The multi-phase personal care composition of claim 1, wherein said multi-phase personal care composition comprises less than about 5%, by weight of said multi-phase personal care composition, of surfactant.

3. The multi-phase personal care composition of claim 2, wherein said multi-phase personal care composition comprises less than about 1%, by weight of said multi-phase personal care composition, of surfactant.

4. The multi-phase personal care composition of claim 3, wherein said multi-phase personal care composition comprises less than about 0.5%, by weight of said multi-phase personal care composition, of surfactant.

5. The multi-phase personal care composition of claim 1, wherein said colorant is an oil-soluble colorant.

6. The multi-phase personal care composition of claim 1, wherein said composition is packaged in a transparent or translucent container.

7. The multi-phase personal care composition of claim 1, wherein said viscosity of said opaque second phase differs from said viscosity of said opaque first phase by no more than 20%.

8. The multi-phase personal care composition of claim 7, wherein said second phase comprises substantially the same components as said first phase.

9. The multi-phase personal care composition of claim 8, wherein a difference between said first phase and said second phase is colorant.

10. The multi-phase personal care composition of claim 1, wherein said first phase is non-Newtonian and said second phase is non-Newtonian.

11. The multi-phase personal care composition of claim 1, wherein said first phase is an oil-in-water emulsion and said second phase is an oil-in-water emulsion.

12. A process of making a multi-phase personal care composition, wherein said process comprises the steps of:
making a first phase comprising an oil/emollient benefit agent and an oil-soluble colorant, wherein said oil/emollient benefit agent and said oil-soluble colorant are mixed to form a premix, wherein said premix is combined with additional optional ingredients to form said first phase;
making a second phase; and
combining said first phase and said second phase to form said multi-phase personal care composition; wherein said multi-phase personal care composition comprises less than about 10%, by weight of said multi-phase personal care composition, of surfactant.

13. A method of moisturizing or conditioning skin or hair, said method comprising the steps of:
wetting said skin or hair with water;
applying to said skin or hair a multi-phase personal care composition of comprising:
a first phase and a second phase; wherein said phases form a visually distinct pattern and wherein said multi-phase personal care composition comprises less than about 10%, by weight of said multi-phase personal care composition, of surfactant; and
rinsing said multi-phase personal care composition from said skin or hair with water.

14. The method of claim 13, wherein said multi-phase personal care composition comprises less than about 5%, by weight of said multi-phase personal care composition, of surfactant.

15. The method of claim 14, wherein said multi-phase personal care composition comprises less than about 1%, by weight of said multi-phase personal care composition, of surfactant.

16. The method of claim 15, wherein said multi-phase personal care composition comprises less than about 0.5%, by weight of said multi-phase personal care composition, of surfactant.

17. The method of claim 13, wherein said colorant is an oil-soluble colorant.

18. The method of claim 13, wherein said composition is packaged in a transparent or translucent container.

19. The method of claim 13, wherein said viscosity of said opaque second phase differs from said viscosity of said opaque first phase by no more than 20%.

20. The method of claim 19, wherein said second phase comprises substantially the same components as said first phase.

21. The method of claim 20, wherein a difference between said first phase and said second phase is colorant.

22. The method of claim 13, wherein said first phase is non-Newtonian and said second phase is non-Newtonian.

23. The method of claim 13, wherein said first phase is an oil-in-water emulsion and said second phase is an oil-in-water emulsion.
